(19) United States
(12) Patent Application Publication
(10) Pub. No.: US 2023/0355558 A1
(43) Pub. Date: Nov. 9, 2023

(54) SYSTEM AND METHOD OF DELIVERING INHIBITORS FOR PREVENTING THE ENTRY OF SARS-CoV-2 AND EMERGING VARIANTS

(71) Applicants: Binoy K. Bordoloi, Jorhat (IN); SACHIN KUMAR, Guwahati (IN); PRIYANKA MAZUMDER, Nadia (IN); Ronen K. Bordoloi, Bridgewater, NJ (US); Rodney L. Eisenberg, Berea, KY (US)

(72) Inventors: Binoy K. Bordoloi, Jorhat (IN); SACHIN KUMAR, Guwahati (IN); PRIYANKA MAZUMDER, Nadia (IN); Ronen K. Bordoloi, Bridgewater, NJ (US); Rodney L. Eisenberg, Berea, KY (US)

(73) Assignee: Bordoloi Biotech LLC, Bridgewater, NJ (US)

(21) Appl. No.: 18/143,942

(22) Filed: May 5, 2023

(30) Foreign Application Priority Data
May 6, 2022 (IN) 202231026507

(51) Int. Cl. A61K 31/195; A61K 45/06; A61P 31/14

(52) U.S. Cl. CPC A61K 31/195; A61K 45/06; A61P 31/14

(57) ABSTRACT

The present invention discloses a composition and method for treating viral infections. The composition and method deliver inhibitors for preventing the entry of SARS-Cov-2 viral infections and the emerging variants are disclosed. The composition comprises a protease inhibitor comprising calcium salt of Tranexamic acid (Ca-TA). The protease inhibitor further comprises lithium salt of Tranexamic acid (Li-TA). The composition demonstrates efficacy in fibrin clot systems, where the Ca salt shows unexpected behavior of clot swelling, unlike TA. This inhibitor blocks the peptide or enzyme known as plasmin from interacting with fibrin (a protein) and subsequently breaking it down. TA is a safe and commonly used small molecule drug. The Ca-TA inhibitor interferes with the binding of the viral spike protein of SARS-CoV-2 to the human cell surface receptor angiotensin-converting enzyme-2 (ACE-2) to avoid infections caused by SARS-CoV-2 infections and emerging variants.

### CROSS-REFERENCE TO RELATED APPLICATION

This application claims priority to Indian Provisional Patent Application No. 202231026507 titled “SYSTEM AND METHOD OF DELIVERING INHIBITORS FOR PREVENTING THE ENTRY OF SARS-CoV-2 AND EMERGING VARIANTS” filed on May 6, 2022. The specification of the above referenced patent applications is incorporated herein by reference in its entirety.

### BACKGROUND OF THE INVENTION

### A. Technical Field

The present invention generally relates to viral infections. More specifically, the present invention relates to a composition and method comprising small molecules as inhibitors for treating and preventing the entry of SARS-Cov-2 viral infections and the emerging variants.

### B. Description of Related Art

SARS is Severe Acute Respiratory Syndrome. The COVID-19 pandemic caused by the severe acute respiratory syndrome coronavirus-2 (SARS-CoV-2) includes at least 272 million cases worldwide, 5.3 million deaths, and over 600 000 new cases daily as of December 2021. Vaccines have been developed, but due to their limited availability and the rate of virus mutation, SARS-CoV-2 infections are likely to continue for many years. As the pandemic continues, several SARS-CoV-2 variants have emerged that are circulating globally, including the variant B.1.1.7 (alpha, first detected in the United Kingdom), variant B.1.351 (beta, first detected in South Africa), and variant B.1.617.2 (delta, first detected in India). These variants of concern are reported to have the capacity to escape humoral immunity elicited by natural infection or the current vaccinations. Moreover, the variants are associated with increases in infections and hospitalizations, suggesting a competitive fitness advantage over the original strain.

A member of the Coronaviridae family, SARS-CoV-2 is an enveloped, nonsegmented, positive sense RNA virus that is characterized by crown-like spikes on the outer surface. SARS-CoV-2 contains RNA strands 29.9 kb long that encode the four main structural proteins, spike, envelope, membrane, and nucleocapsid, 16 nonstructural proteins, and several accessory proteins. The novel coronavirus is challenging scientists as they track its spread locally and globally. World leaders are enforcing social-distancing measures, wearing of face masks, frequent washing of hands, and use of hand sanitizers and disinfectants to prevent the spread of the coronavirus.

The SARS-CoV-2 pandemic has laid bare the urgent need for a better understanding of how viruses jump from animals to people. The most widely held hypothesis is that viruses derive from the bits of nucleic acid that “escaped” from cellular organisms. However, viruses are parasites, dependent on their hosts. Some viruses infect animal cells and others infect plant cells. The biology and chemistry of a virus are explained below first so that the latter sections on the science of its testing, treatment, and prevention will make more sense.

A virus is a tiny, infectious particle consisting of a nucleic acid core (its genetic material) surrounded by a protein called a capsid. Some viruses are also surrounded by an outer membrane envelope containing proteins, lipids, carbohydrates, and traces of metals. A typical small virus such as poliovirus is 20 nm (nano-meter) in diameter, whereas a larger virus such as the poxvirus that causes smallpox, maybe 400 nm long and 200 nm wide. The SARS-CoV-2 dimension is also in nm. Viruses are acellular and cannot independently perform metabolic activities. They do not have the components to carry on cellular respiration or synthesize proteins and other molecules. Other living organisms contain both DNA (deoxyribo-nucleic acid) and RNA (ribo-nucleic acid). But a virus contains either DNA or RNA, but not both. Viruses reproduce, but only within the complex environment of the living cell they infect. They use their genetic information to force the host cell to replicate the viral nucleic acid and to take over the mechanisms of the host cell. The host cell then synthesizes the capsid and the envelope components of the virus. An average-sized protein, a polypeptide, is about 360 amino-acids long, which can be assembled by a bacterium in about 18 seconds, and by a eukaryotic cell in a little over 1 minute.

Nucleic acids (first discovered in 1870) transmit hereditary information and determine what proteins are manufactured by the cell. There are two classes of nucleic acids, DNA and RNA. DNA comprises the genes, the hereditary material of the cell, and contains instructions for making all the proteins, as well as the RNA the organism needs. Nucleic acids are polymers (poly=many, mer=unit, for example, poly-ester) of nucleotides. A nucleotide is a molecule consisting of (1) a five-carbon sugar (either ribose in RNA or deoxyribose in DNA), (2) one or more phosphate groups that make the molecule acidic, and (3) a nitrogenous base, which is a ring compound that contains nitrogen (purine or pyrimidine). DNA is “transcribed” (kind of “copying”) to form RNA. RNA is “translated” to form a polypeptide. mRNA (Messenger RNA) is a single, uncoiled strand of RNA that carries the specific information for making a 2 protein. Although many polypeptide chains can be actively synthesized on a single mRNA at any one time, the half-life of an mRNA molecule itself in bacterial cells is only about 2 minutes. (Half-life is the time it takes for half the molecules to be degraded). Eukaryotic (meaning cells with nucleus, and membrane-bound materials, for example, fungi, plants and animals) mRNAs have half-lives ranging from 30 minutes to as long as 24 hours, the average in mammalian cells being about 10 hours.

FIG. 1 shows SARS-Cov-2 100 with unique spike protein. Unlike human genetic information, which is enclosed in double-stranded DNA, the new coronavirus, like all coronaviruses, stores the genetic information in a single strand of RNA. The human genome contains around 3 billion base pairs tightly packed inside the nucleus of each of the human cells. In contrast, the coronavirus's RNA genome has fewer than 30,000 bases. This shorter sequence codes for the 29 proteins that make up the virus, now dubbed SARS-CoV-2. The scientific challenge is how to stop this entity of “29,905 RNA bases in a lipid, protein and sugar shell”.

Its RNA is contained in a shell decorated with protein spikes. After entering the body, the virus lodges in the respiratory tract. These spikes allow the virus to bind to receptors in the human cell. Once inside, the virus uses mechanisms in the cell to make copies of itself. These copies are released into the body to infect other cells. FIG. 2 shows the basic concept of developing vaccine 200 using the concept of antigen 202, antibodies 204, and neutralized antigen 206. Different antibodies, for example, IgM, IgG, have different production start times and duration in the blood. The human body's immune system detects the antigen (infectious agent) 202, it releases antibodies 204 and provides a neutralized antigen 206 as shown in FIG. 2. These bind to the antigen 202, neutralizing it or marking it to be destroyed by immune cells. The human body's immune system's memory cells “remember” previous antigens. If a person is infected again, the cells rapidly generate antibodies 204 to get rid of the antigens. In the case of COVID-19, however, the human antibodies may not attack the virus, because they cannot bind strongly to its spike proteins.

The therapies for COVID-19 can be broadly categorized as (a) new vaccines, (b) convalescent plasma, (c) NK-cell therapy, (d) monoclonal antibodies, and (e) antiviral drugs, and (f) herbals and others. One vaccine, now in the human clinical trial, injects a portion of the virus's genetic material into the body. Cells then produce these viral fragments and antibodies, learn to recognize them for when an actual virus attacks. Antivirals work in different ways. Some can block a virus from infecting human cells. Others are made from antibodies that are isolated from infected people who recover.

Any step of the SARS-CoV-2 virus infection and replication cycle is a potential target for antiviral intervention including cell entry, genome replication, viral maturation, or viral release. However, the binding of the viral spike protein of SARS-CoV-2 to the human cell surface receptor angiotensin-converting enzyme-2 (ACE2) is a critical step during the infection of human cells. Therefore, cell entry inhibitors could be used to prevent SARS-CoV-2 infection as well as to shorten the course of COVID-19 infections by preventing virus particles from infecting human cells.

The vaccines commonly use inactivated viruses or antigen fragments to imitate infection without causing disease. The vaccine triggers an immune response so that the human immune system's memory cells recognize the virus or other antigen if the person is ever infected. For some diseases, vaccine-induced immunity lasts a lifetime. For others, it fades over time. For example, flu viruses mutate, meaning that existing antibodies don't recognize them and yearly vaccination is needed.

In addition, there are various approved antiviral agents showing efficacy for the treatment or prevention of coronavirus infections, such as SARS-CoV-2. However, in view of the outbreak and its toll on human lives, there is a need for improved therapeutic options for treating coronavirus infections, especially those caused by SARS-CoV-2.

### SUMMARY OF THE INVENTION

The present invention generally discloses a composition and method for treating viral infections. The composition is for treating, preventing or reducing symptoms of viral infections, such as those caused by SARS-CoV-2. Further, the composition comprises small molecules as inhibitors for treating and preventing the entry of SARS-Cov-2 viral infections.

In one embodiment, the present invention discloses a composition comprising a protease inhibitor comprising calcium salt of Tranexamic acid (Ca-TA). The protease inhibitor further comprises lithium salt of Tranexamic acid (Li-TA). In another embodiment, the present invention discloses a composition comprising a protease inhibitor comprising at least one of calcium salt of Tranexamic acid (Ca-TA) and lithium salt of Tranexamic acid (Li-TA). In yet another embodiment, the present invention discloses a composition comprising a combination of calcium salt of Tranexamic acid (Ca-TA) and lithium salt of Tranexamic acid (Li-TA).

In one embodiment, the composition further comprises a therapeutic agent comprising antibody, and peptides. In another embodiment, the composition further comprises a therapeutic agent comprising antibody, peptides, small drug molecules, Alzheimer's drug molecules, and combination thereof. In one embodiment, the calcium salt of Tranexamic acid is in the range of 10 to 30 mM. In another embodiment, the lithium salt of Tranexamic acid is in the range of 10 to 30 mM. The composition is formulated for treating acute respiratory syndrome coronavirus-2 (SARS-CoV-2) and for treating emerging variants of acute respiratory syndrome coronavirus-2 (SARS-CoV-2).

The present invention further discloses a method of treating viral infection. The method comprising the step of administering a sustainable release of composition of a protease inhibitor comprising calcium salt of Tranexamic acid (Ca-TA). The protease inhibitor further comprises lithium salt of Tranexamic acid (Li-TA). In another embodiment, the composition comprises a protease inhibitor comprising at least one of calcium salt of Tranexamic acid (Ca-TA) and lithium salt of Tranexamic acid (Li-TA). In yet another embodiment, the composition comprises a combination of calcium salt of Tranexamic acid (Ca-TA) and lithium salt of Tranexamic acid (Li-TA).

In yet another embodiment, the composition further comprises a therapeutic agent comprising antibody, and peptides. In yet another embodiment, the composition further comprises a therapeutic agent comprising antibody, peptides, small drug molecules, Alzheimer's drug molecules, and combination thereof. In one embodiment, the calcium salt of Tranexamic acid is in the range of 10 to 30 mM. In another embodiment, the lithium salt of Tranexamic acid is in the range of 10 to 30 mM.

According to the present invention, improved therapeutic options are utilized for treating coronavirus infections, especially those caused by severe acute respiratory syndrome coronavirus-2 (SARS-CoV-2) and its variants, which cause COVID-19. In an exemplary embodiment, a therapeutic small molecule is used as inhibitor for treating and preventing the entry of SARS-Cov-2 viral infections and the emerging variants, and other viral infections. In one embodiment, the small molecule may be Calcium salt of Tranexamic acid (Ca-TA) as a protease inhibitor and its sustained release. It demonstrates efficacy in fibrin clot systems, where the Ca salt shows unexpected behavior of clot swelling unlike TA, and so on. This inhibitor blocked the peptide or enzyme known as plasmin from interacting with fibrin (a protein) and subsequently breaking it down.

The Ca-TA demonstrates a mechanism that the spike protein C-terminal domain interface contributes to a network of “hydrogen bonding and salt bridge interactions” with the ACE-2 receptor. The TMPRSS2 protease on the host cell membrane activates the spike protein by cleaving it at S1/S2 sites leading to conformational changes that allow the virus to fuse with the host membrane and enter the cell. The Ca-TA with any efficacy to SARS-Cov-2 system interferes with the binding of the viral spike protein of SARS-CoV-2 to the human cell surface receptor angiotensin converting enzyme-2 (ACE-2)

The infection of host cells by SARS-CoV-2 begins with the attachment of the receptor-binding domain (RBD) of the S1 protein, which has been identified as residues 331 to 524, to the host cell receptor ACE2. An enzyme on the outer cell membrane of host cells, ACE2 is expressed abundantly on human endothelial cells in the lungs, arteries, heart, kidney, and intestines. TMPRSS2 protease on the host cell membrane activates the spike protein by cleaving it at S1/S2 and S2 sites, leading to conformational changes that allow the virus to fuse with the host membrane and enter the cytoplasm. The S1 subunit is primarily responsible for the determination of the host virus range and cellular tropism. The Ligands with high affinity to the receptor binding domain on the S1 protein have the potential to function as entry inhibitors and prevent infection of human cells by SARS-CoV-2. For example, small peptides derived from the heptad repeat regions of SARS-CoV-1 spike S2 subunit have been utilized to inhibit SARS-CoV-2 infection by the interference of fusion with target cells. The approach of utilizing compounds that block virus-receptor interaction has also been useful for other viruses, including HIV-1 and hepatitis C virus. Further, natural products are the most successful source of drugs and drug leads in the history of pharmacology.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 shows SARS-Cov-2 has a unique spike protein, main protease, and RNA-dependent RNA polymerase in its proteins.

FIG. 2 shows a concept of antigen, antibodies, and neutralized antigen for developing a vaccine.

FIG. 3A shows a comparison chart of NMR spectra of Ca-TA and TA in one embodiment of the present invention.

FIG. 3B shows the chemical structure of TA in one embodiment of the present invention.

FIG. 3C shows the chemical structure of Ca-TA in one embodiment of the present invention.

FIG. 3D shows the chemical structure of MTA in one embodiment of the present invention.

FIG. 4A shows a visual observation of the representation of the degradation of fibrin clots by plasmin with varying concentrations of TA in one embodiment of the present invention.

FIG. 4B shows a visual observation of the representation of the degradation of fibrin clots by plasmin with varying concentrations of Ca-TA in one embodiment of the present invention.

FIG. 5A shows a graphical representation of quantification of the degradation of fibrin clots by plasmin with varying concentrations of TA in triplicate in one embodiment of the present invention.

FIG. 5B shows a graphical representation of quantification of the degradation of fibrin clots by plasmin with varying concentrations of Ca-TA in triplicate in one embodiment of the present invention

FIG. 6 shows a graph having a comparison of fibrin clot degradation of Ca-TA versus TA in one embodiment of the present invention.

FIG. 7 shows a graph of quantification of the degradation of fibrin clots by plasmin with varying concentrations of TA and MTA in one embodiment of the present invention.

FIG. 8A shows a visual observation of the representation of the degradation of fibrin clots by plasmin with varying concentrations of TA and MTA at zero hours (0 h) in one embodiment of the present invention.

FIG. 8B shows a visual observation of the representation of the degradation of fibrin clots by plasmin with varying concentrations of TA and MTA at 16 hours in one embodiment of the present invention.

FIG. 9A shows a graph of an overdose of a drug during cellular toxicity assay in one embodiment of the present invention.

FIG. 9B shows a graph of cellular viability during cellular toxicity assay in one embodiment of the present invention.

FIG. 10 exemplarily illustrates a nuclear magnetic resonance (NMR) spectrum of tranexamic acid.

FIG. 11 exemplarily illustrates a nuclear magnetic resonance (NMR) spectrum of tranexamic acid, lithium tranexamate and calcium tranexamate.

FIG. 12 is a representation of a plaque assay of TA, MTA and Ca-TA at various concentration on cells infected with virus.

FIG. 13 is a graph illustrating an activity of TA, MTA and Ca-TA at various concentration against the virus infected cells.

FIG. 14 exemplarily illustrates a representation of an antiviral plaque assay of Ca-TA at various concentration against the virus infected cells.

FIG. 15 is a graph illustrating an activity of Ca-TA at various concentration against the virus infected cells.

FIG. 16 exemplarily illustrates a representation of an antiviral plaque assay of Li-TA at various concentration on cells infected with virus.

FIG. 17 is a graph illustrating analysis of swelling effect for Ca-TA and Li-TA.

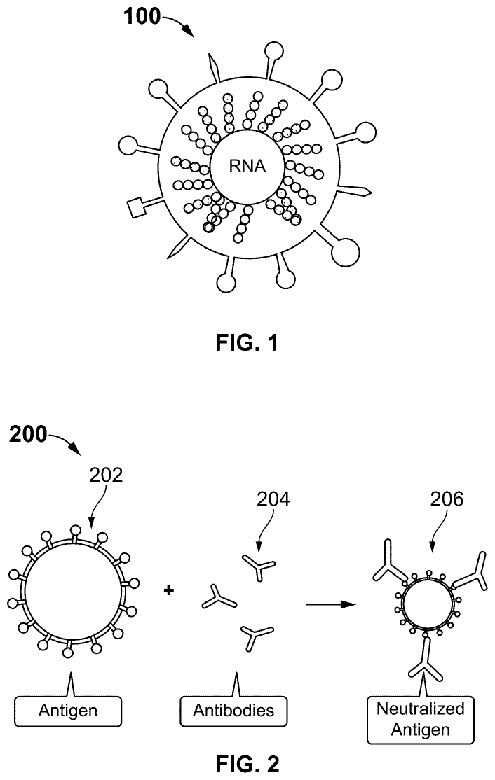

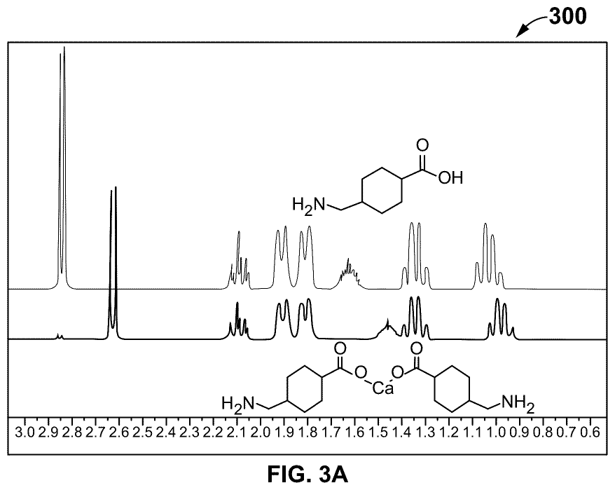

### DETAILED DESCRIPTION OF EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

The present invention generally discloses a composition and method for treating viral infections. The composition is for treating, preventing or reducing symptoms of viral infections, such as those caused by SARS-CoV-2. Further, the composition comprises small molecules as inhibitors for treating and preventing the entry of SARS-Cov-2 viral infections.

In one embodiment, the present invention discloses a composition comprising a protease inhibitor comprising calcium salt of Tranexamic acid (Ca-TA). The protease inhibitor further comprises lithium salt of Tranexamic acid (Li-TA). In another embodiment, the present invention discloses a composition comprising a protease inhibitor comprising at least one of calcium salt of Tranexamic acid (Ca-TA) and lithium salt of Tranexamic acid (Li-TA). In yet another embodiment, the present invention discloses a composition comprising a combination of calcium salt of Tranexamic acid (Ca-TA) and lithium salt of Tranexamic acid (Li-TA). In yet another embodiment, the protease inhibitor comprises zinc salt of Tranexamic acid (Zn-TA).

In one embodiment, the composition further comprises a therapeutic agent comprising antibody, and peptides. In another embodiment, the composition further comprises a therapeutic agent comprising antibody, peptides and small drug molecules. The composition of the present invention could be used with other therapeutic agents, for example, the therapeutic agent of Alzheimer's drug molecule comprising antibody, peptides and small drug molecules. In yet another embodiment, the composition further comprises a therapeutic agent comprising antibody, peptides, small drug molecules, Alzheimer's drug molecules, and combination thereof. In one embodiment, the calcium salt of Tranexamic acid is in the range of 10 to 30 mM. In another embodiment, the lithium salt of Tranexamic acid is in the range of 10 to 30 mM. The composition is formulated for treating acute respiratory syndrome coronavirus-2 (SARS-CoV-2) and for treating emerging variants of acute respiratory syndrome coronavirus-2 (SARS-CoV-2). In yet another embodiment, the composition comprises lithium citrate. In yet another embodiment, the therapeutic agent includes lithium compounds. The composition of the present invention comprising lithium compounds could be used for treatment of depression and bipolar disorder. In yet another embodiment, the composition further comprises bismuth salt of Tranexamic acid.

The present invention further discloses a method of treating viral infection. The method comprising the step of administering a sustainable release of composition of a protease inhibitor comprising calcium salt of Tranexamic acid (Ca-TA). The protease inhibitor further comprises lithium salt of Tranexamic acid (Li-TA). In another embodiment, the composition comprises a protease inhibitor comprising at least one of calcium salt of Tranexamic acid (Ca-TA) and lithium salt of Tranexamic acid (Li-TA). In yet another embodiment, the composition comprises a combination of calcium salt of Tranexamic acid (Ca-TA) and lithium salt of Tranexamic acid (Li-TA).

In yet another embodiment, the composition further comprises a therapeutic agent comprising antibody, and peptides. In yet another embodiment, the composition further comprises a therapeutic agent comprising antibody, peptides, small drug molecules, Alzheimer's drug molecules, and combination thereof. In one embodiment, the calcium salt of Tranexamic acid is in the range of 10 to 30 mM. In another embodiment, the lithium salt of Tranexamic acid is in the range of 10 to 30 mM.

In yet another embodiment, the composition comprises lithium citrate. The composition may comprise small quantities of lithium citrate. In yet another embodiment, the therapeutic agent includes lithium compounds. The lithium compounds are used as a treatment for depression and bipolar disorder. In yet another embodiment, the composition comprises bismuth salt of Tranexamic acid.

According to the present invention, the composition and method provides an improved therapeutic option that are utilized for treating coronavirus infections, especially those caused by severe acute respiratory syndrome coronavirus-2 (SARS-CoV-2) and its variants, which cause COVID-19. In an exemplary embodiment, a therapeutic small molecule is used as inhibitor for treating and preventing the entry of SARS-Cov-2 viral infections and the emerging variants, and other viral infections. In one embodiment, the small molecule may be Calcium salt of Tranexamic acid (Ca-TA) as a protease inhibitor and its sustained release. It demonstrates efficacy in fibrin clot systems, where the Ca salt shows unexpected behavior of clot swelling unlike TA, and so on. This inhibitor blocked the peptide or enzyme known as plasmin from interacting with fibrin (a protein) and subsequently breaking it down. TA is a safe and commonly used small molecule drug. Further, Ca-TA and Li-TA proves better in their protease inhibition activity, particularly as related to stopping bleed (more specifically, for treating cerebral bleeding, particularly in Alzheimer's disease).

The Ca-TA demonstrates a mechanism that the spike protein C-terminal domain interface contributes to a network of “hydrogen bonding and salt bridge interactions” with the ACE-2 receptor. The TMPRSS2 protease on the host cell membrane activates the spike protein by cleaving it at S1/S2 sites leading to conformational changes that allow the virus to fuse with the host membrane and enter the cell. In one embodiment, the Ca-TA has any efficacy with SARS-Cov-2 system where the inhibitor interferes with the binding of the viral spike protein of SARS-CoV-2 to the human cell surface receptor angiotensin converting enzyme-2 (ACE-2).

The infection of host cells by SARS-CoV-2 begins with the attachment of the receptor-binding domain (RBD) of the S1 protein, which has been identified as residues 331 to 524, to the host cell receptor ACE2. An enzyme on the outer cell membrane of host cells, ACE2 is expressed abundantly on human endothelial cells in the lungs, arteries, heart, kidney, and intestines. TMPRSS2 protease on the host cell membrane activates the spike protein by cleaving it at S1/S2 and S2 sites, leading to conformational changes that allow the virus to fuse with the host membrane and enter the cytoplasm. The S1 subunit is primarily responsible for the determination of the host virus range and cellular tropism. The Ligands with high affinity to the receptor binding domain on the S1 protein have the potential to function as entry inhibitors and prevent infection of human cells by SARS-CoV-2. For example, small peptides derived from the heptad repeat regions of SARS-CoV-1 spike S2 subunit have been utilized to inhibit SARS-CoV infection by the interference of fusion with target cells. The approach of utilizing compounds that block virus receptor interaction has also been useful for other viruses, including HIV-1 and hepatitis C virus. Further, natural products are the most successful source of drugs and drug leads in the history of pharmacology.

### Example Preparation Method of Calcium Tranexamate and Characterization

Calcium tranexamate is prepared from the potassium salt of tranexamic acid and calcium nitrate in methanol. The 5-proton of calcium tranexamate is diagnostic as a distinctive signal at 1.46 ppm (ttt, J=11.4, 7.6, 3.8) appearing as a complex multiplet, significantly downfield from its counterpart in tranexamic acid which is zwitterion. The charged amino group influence the protons of the 6.4 and 4 enough to move their resonance value downfield relative to the free base that is found in the calcium tranexamate. Careful titration of a mixture of calcium nitrate and tranexamic acid in methanol with potassium hydroxide should yield a pure form of the much less soluble calcium tranexamate. Washing with methanol should provide substantially pure calcium tranexamate.

Calcium bis-tranexamate proton NMR is provided as follows.

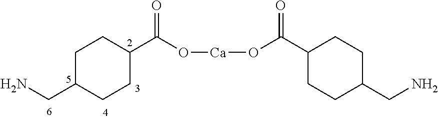

1H-NMR (400 MHz, DO): 2.62 (d, J=6.8 Hz, 2H), 2.09 (tt, J=12.3, 3.5 Hz, 1H), 1.90 (dd, J=13.2, 2.6 Hz, 2H), 1.81 (dd, J=13.2, 2.7 Hz, 2H), 1.51-1.41 (m, 1H), 1.34 (qd, J=12.8, 3.2 Hz, 2H), 0.98 (qd, J=12.6, 3.3 Hz, 2H).

Tranexamic acid proton NMR is provided as follows.

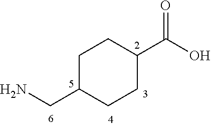 [SMILES: NCC1CCC(C(=O)O)CC1]

1H-NMR (400 MHz, D20): 2.84 (d, J=7.1 Hz, 4H), 2.09 (tt, J=12.3, 3.5 Hz, 2H), 1.91 (dd, J=13.4, 2.6 Hz, 4H), 1.81 (dd, J=13.1, 2.3 Hz, 4H), 1.63 (ttt, J=11.4, 7.6, 3.8 Hz, 2H), 1.34 (qd, J=12.8, 3.2 Hz, 4H), 1.03 (qd, J=12.6, 3.3 Hz, 4H).

Another example preparation method of calcium tranexamate is disclosed as follows. Calcium bis-tranexamate is prepared from a methanolic solution of calcium nitrate and a solution of sodium tranexamate which was itself prepared from tranexamic acid and solid sodium hydroxide dissolved in anhydrous methanol. A white powder slowly appears, which is precipitated upon stirring overnight at room temperature. Proton NMR analysis of the isolated and dried white powder showed no starting tranexamic acid, or sodium tranexamate but a new species that has the same proton NMR splitting pattern as tranexamic acid but with different ppm values. A comprehensive characterization is still in progress. In a qualitative test for relative solubility of the putative calcium salt, the powder was dissolved in a minimum of water, and then an identical amount in pH 7.4 100 mM phosphate buffer. The buffered solution requires approximately four times as much solvent to dissolve the material as with water. The material was not soluble to a significant degree in either methanol or propanol at room temperature.

### Example Preparation Method of Lithium Tranexamate Monohydrate

Tranexamic acid (5.000 g, 0.0318 mol) was dissolved in deionized water (34 mL) at room temperature, and lithium monohydrate (0.764 g, 0.0318 mol) in water (5 mL). The solutions are added and stirred under Ar for 2 h. The solution was dried in vacuo and then suspended in anhydrous ethanol and filtered. The resulting powder was dried at room temperature for several days under high vacuum. The white powdery solid (3.25 g) was analyzed by 1H-NMR (500 MHz, D20), by combustion analysis, and by flame photometry (for lithium content). The analytical results are provided as follows. The empirical formula is C₈H₁₄NO₂Li·½ H₂O, the formula weight of about 172.15 and chemical purity is >99.7% (H-NMR, 500 MHz in D20).

### Example Preparation Method of Zinc Tranexamate Mono Hydrate

Tranexamate acid (6.32 g, 40 mmol) is dissolved in methanol (65 mL) and stirred at 50° C. until clear. To the stirring solution, a solution of NaOH (1.60 g, 40 mmol) in MeOH (8 mL) and the solution stirred 30 min at 50° C. is added. The solution was cooled to room temperature whereupon a solution of zinc acetate dihydrate (4049 g, 20 mmol) in methanol (50 mL) added dropwise over about 10 min. The solution was stirred for approximately 4 h then vacuum filtered. The white solid was slurried in methanol (75 mL) and vacuum filtered. The filtered solid was slurried in an identical fashion for few times to remove acetate impurities. After drying at room temp under high vacuum or several days, a white powder was obtained. Proton NMR revealed less than 0.3% acetate and no detectable methanol. The material was characterized by proton and Carbon NMR, combustion analysis and spectrophotometric determination of zinc. The empirical formula is C₁₆H₂₆N₂O₄Zn·H2O, the formula weight of about 393.78 and the chemical purity is −99.7% (1 h-nmr, 500 MHz in D2O).

### Experiment 1

Tranexamic acid (TA) is an antifibrinolytic drug. TA has found application with fibrin sealants in surgical procedures in leak prevention. TA is known for its adverse side effects in high oral dosage. Its neurotoxicity prevents its use with fibrin sealants in neuro-surgical procedures. Therefore, an investigation is undertaken to make derivatives of TA at its acid and amine functional groups, such as, calcium salt of TA (Calcium Tranexmate, Ca-TA), and an amide derivative, named MTA (Modified TA, which is Methoxy Acetic Acid Amido derivative of Tranexamic Acid). This study reports the preliminary efficacy data on Ca-TA and MTA in their respective protease inhibition activities in a proteolytic environment. Synthesized Ca-TA and MTA are used in this experiment as protease inhibitors. The Ca-TA and MTA are water soluble compound. A gravimetric method is used for fibrin clot degradation profiles at pH 7.4 and 37° C. in a proteolytic medium containing plasmin. Longevity of clot under these conditions as compared to the control with no protease inhibitor is used as a measure of efficacy for protease inhibition of Ca-TA and MTA.

### Results and Discussion of Experiment 1:

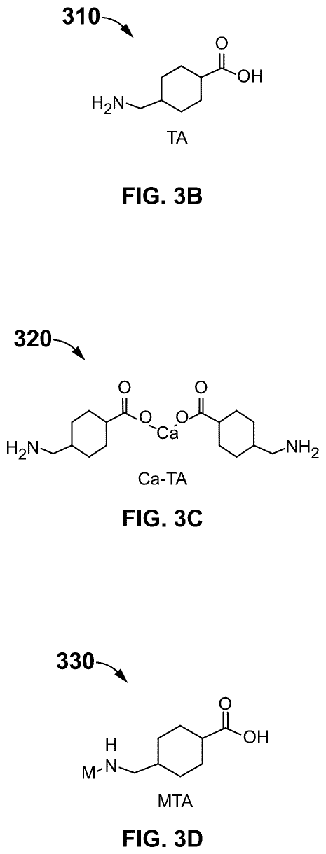

Referring to FIG. 3A, a comparison chart 300 of NMR spectra of Ca-TA (Calcium Tranexamate) and TA (tranexamic acid), according to one embodiment of the present invention. The molecular identity and structures of the Ca-TA and TA are determined using Nuclear magnetic resonance spectroscopy (NMR spectra). The comparison chart 300 has an upper plot showing TA in D20 solvent and a lower plot showing Ca-TA in D20 solvent. FIGS. 3B-3D exemplarily illustrates the chemical structures of the molecules such as TA 310, Ca-TA 320, and MTA (Modified TA) 330, according to one embodiment of the present invention. The molecules are used for fibrin clot degradation at varying concentrations.

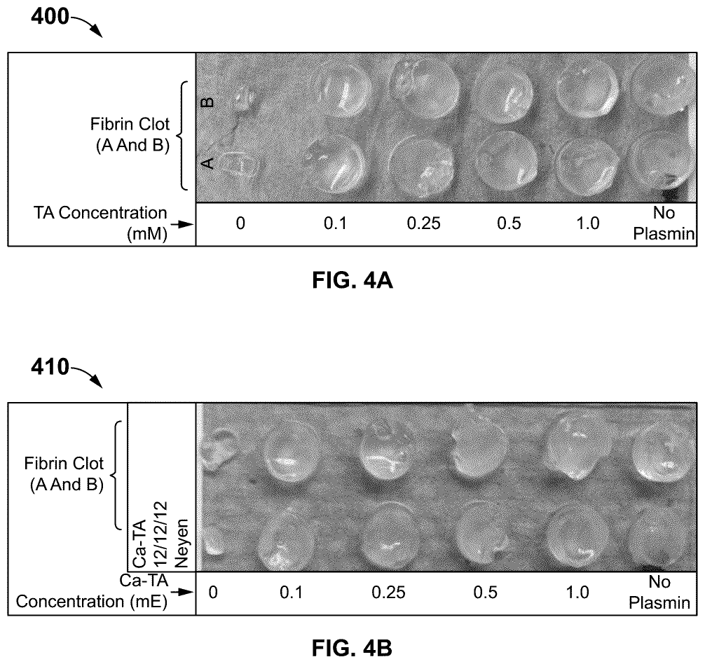

Referring to FIGS. 4A-4B, visual observation of the representation of the degradation of fibrin clots (400 and 410) by plasmin with varying concentrations of TA and Ca-TA in one embodiment of the present invention. The longevity of fibrin clot with varying concentrations of TA (as shown in FIG. 4A) and Ca-TA (as shown in FIG. 4B) in plasmin-containing medium. The degradation of fibrin clots by plasmin is at about 7.4 pH at 37° C. with varying concentrations of TA and Ca-TA. The Fibrin clots with varying concentration of about 0-1 mM of TA and Ca-TA are subjected to degradation by plasmin in PBS buffer. Ca-TA shows dose-dependent protease inhibition activity by inhibiting plasmin mediated degradation of fibrin clots for up to 48 hours.

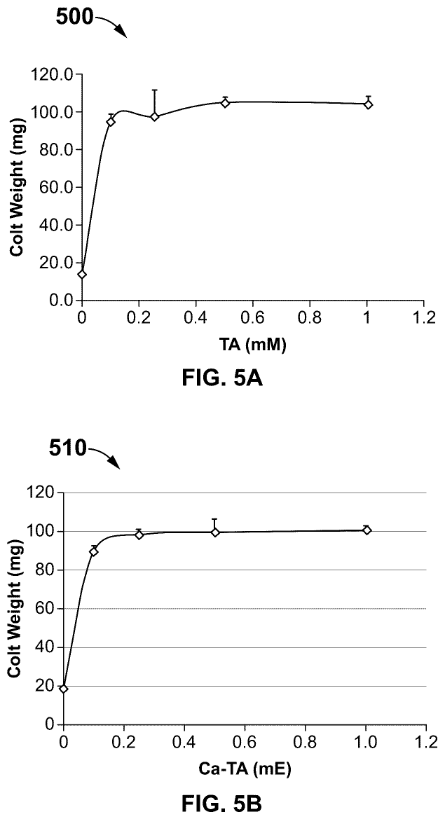

FIGS. 5A-5B exemplarily illustrate graphical representation (500 and 510) of quantification of the degradation of fibrin clots by plasmin at varying concentrations of TA and Ca-TA in triplicate, according to one embodiment of the present invention. The degradation of fibrin clots by plasmin is at 7.4 pH at 37° C. with varying concentrations of TA and Ca-TA in triplicate. TA and Ca-TA provide effective resistance against fibrin clot degradation by plasmin up to 48 hours at about 0.1 mM concentration. The efficacy of Ca-TA is demonstrated and compared with that of TA. Ca-TA is efficacious at a concentration as low as 0.1 mM or below, comparable to that of TA at 0.1 mM.

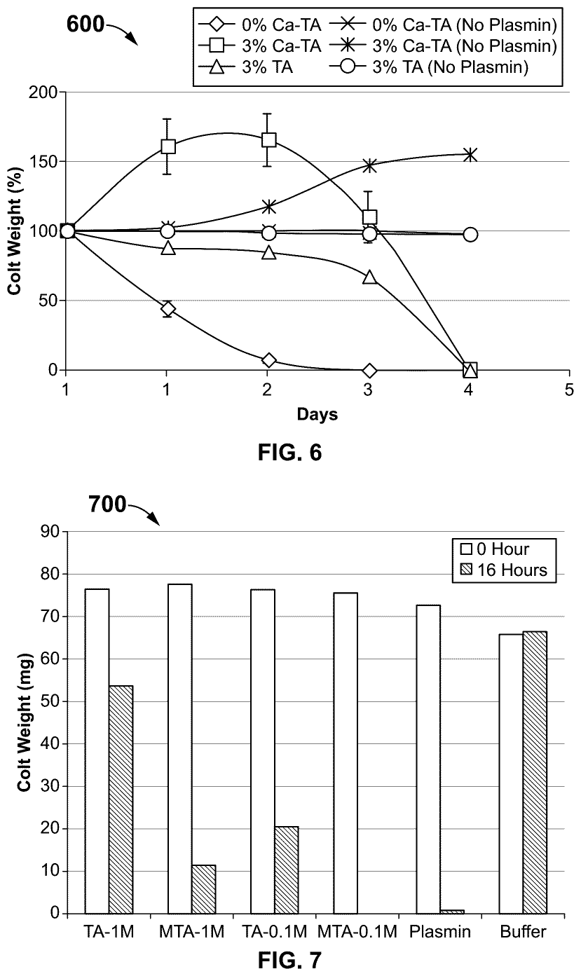

Referring to FIG. 6, a graph 600 has a comparison of fibrin clot degradation of Ca-TA versus TA, according to one embodiment of the present invention. The graph 600 shows about 3% Ca-TA in fibrin clots displaying 50-70% swelling in size compared to TA at the same loading percentage (%). Both Ca-TA and TA at this loading level show inhibited plasmin-mediated degradation of the fibrin clots for a longer period. The fibrin clot degradation of Ca-TA versus TA shows; (a) in a non-degrading medium with no plasmin, while clots with 3% TA did not swell, those with 3% Ca-TA showed more than 50% swelling, and (b) in a degrading medium containing plasmin, while clots with 3% TA degraded without swelling, those with 3% Ca-TA showed more than 50% swelling prior to degradation. Thus Ca-TA indicates potentially superior hemostatic ability as compared to TA.

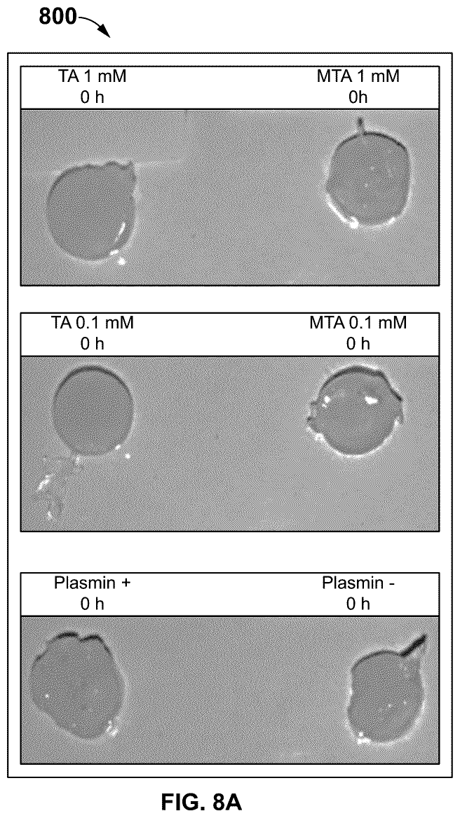

Referring to FIG. 7, a graph 700 of quantification of the degradation of fibrin clots by plasmin with varying concentrations of TA and MTA, according to one embodiment of the present invention. The degradation of fibrin clots by plasmin is at 7.4 pH at 37° C. with varying concentrations of TA and MTA. The graph 700 shows the longevity of fibrin clots with varying concentrations of TA and MTA in plasmin-containing medium. Fibrin clots with varying concentration, for example, from about 1.0 mM to about 0.1 mM of TA and MTA are subjected to degradation by plasmin in PBS buffer. MTA at concentrations of 0.1 mM and 1 mM are evaluated in a proteolytic medium of plasmin for fibrin clot degradation over a duration of about 16 hours at 7.4 pH and 37° C. While 0.1 mM MTA shows 100% degradation of the clot, 1 mM shows degradation of about 85.1+/−0.6%, indicating activity.

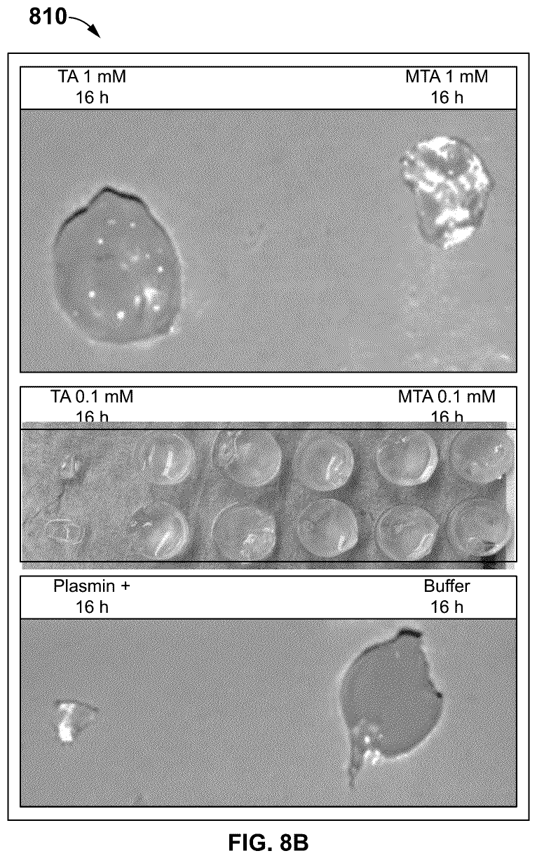

Referring to FIGS. 8A-8B, visual observation (800 and 810) of the representation of the degradation of fibrin clots by plasmin with varying concentrations of TA and MTA, according to one embodiment of the present invention. The degradation of fibrin clots by plasmin is at 7.4 pH at 37° C. with varying concentrations of TA and MTA. MTA at concentrations of about 0.1 mM and 1 mM are evaluated in a proteolytic medium of plasmin for fibrin clot degradation over a duration of about 16 hours at 7.4 pH and 37° C. While 0.1 mM MTA shows 100% degradation of clots, 1 mM shows degradation of about 85.1+/−0.6%, indicating activity.

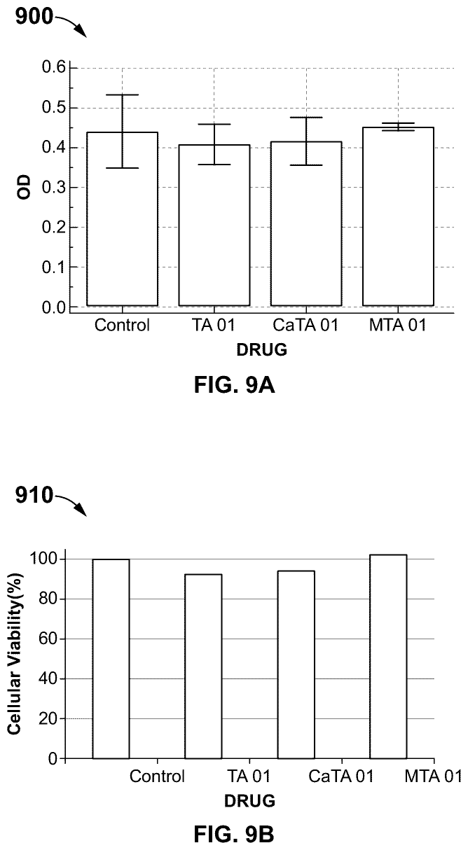

Referring to FIGS. 9A-9B, comparison charts showing neurotoxicity assay of TA, Ca-TA, and MTA. FIG. 9A shows a graphical representation 900 to measure OD at 570 nM of a drug during cellular toxicity assay (MTT ASSAY), according to one embodiment of the present invention. Cells are treated with 1 ug/mL of TA, Ca-TA, and MTA at about 570 nM concentration. FIG. 9B shows a graph 910 of cellular viability during cellular toxicity assay, according to one embodiment of the present invention. It shows the cellular viability (%) of the cells that are treated with 1 ug/mL of TA, Ca-TA, and MTA.

Human neuroblastoma cell line Sh-Sy5y are cultured in DMEM supplemented with about 10% FBS. 50000 cells are seeded in 96 well plate per well. The cells are serum starved for 24 hours before treatment. The cells are then treated with 1 ug/mL of TA, Ca-TA, and MTA. Untreated cells are kept as control. After 24 hours of treatment, about 0.1 mg/mL of MTT reagent is added to each well and kept for about 3.5 hours in a CO₂-incubator. The resulting formazan crystals are dissolved in DMSO. Absorbance is measured using a plate reader at 570 nm. Readings from blank are subtracted from the control and treated wells.

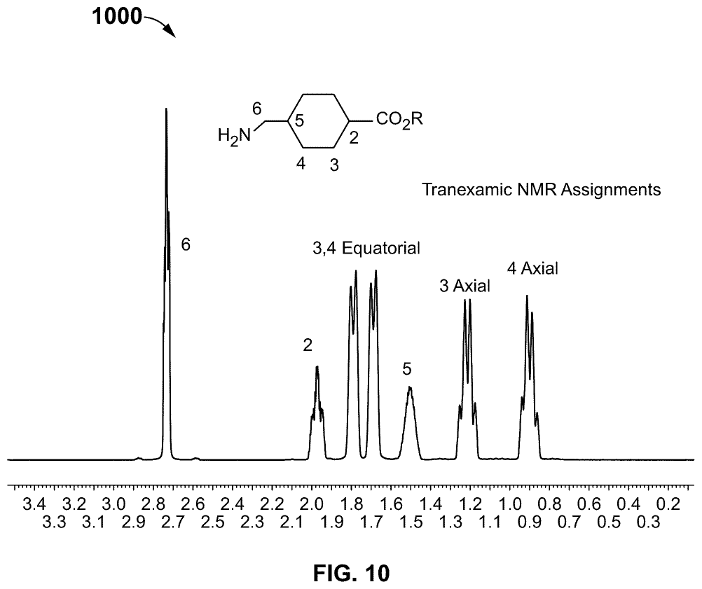

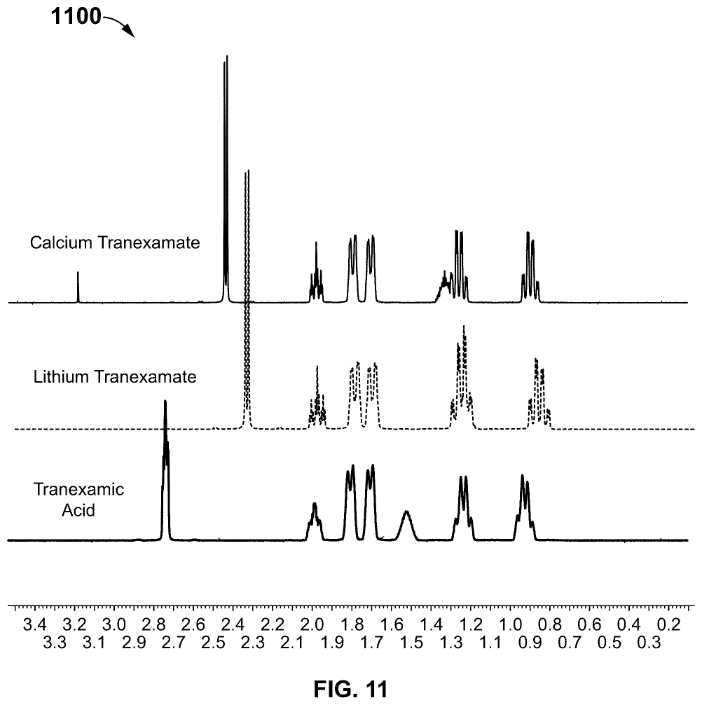

FIG. 10 exemplarily illustrates a nuclear magnetic resonance (NMR) spectrum 1000 of tranexamic acid. FIG. 11 exemplarily illustrates a nuclear magnetic resonance (NMR) spectrum 1100 of tranexamic acid, lithium tranexamate and calcium tranexamate. Referring to FIG. 10 and FIG. 11, viral plagues are reduced with the increase in concentration of lithium tranexamate and calcium tranexamate.

### Experiment 2

This section discloses an experimental procedure to check the antiviral activity of Tranexamic acid (TA), N-(methoxy-acetamido) tranexamic acid (MTA), Ca-TA, and Li-TA. Cells were seeded in 12 well plate and kept the plate in incubator at 37° C., 5% CO2 for 14-16 hr until it reaches to the appropriate confluency. Then the cells were infected with virus (NDV) diluted in cell culture media and incubated for 1 hr for viral adsorption. Compounds were prepared in different concentration in overlay media (methyl cellulose). After that, cells were washed with PBS and overlay media was added to the cells. The cells were incubated 48-72 hrs based on the plaque formation in the monolayer of the cells. When the plaque was formed, the overlay media was removed and the monolayer was fixed with methanol followed by crystal violet staining for further examination.

### Results of Experiment 2:

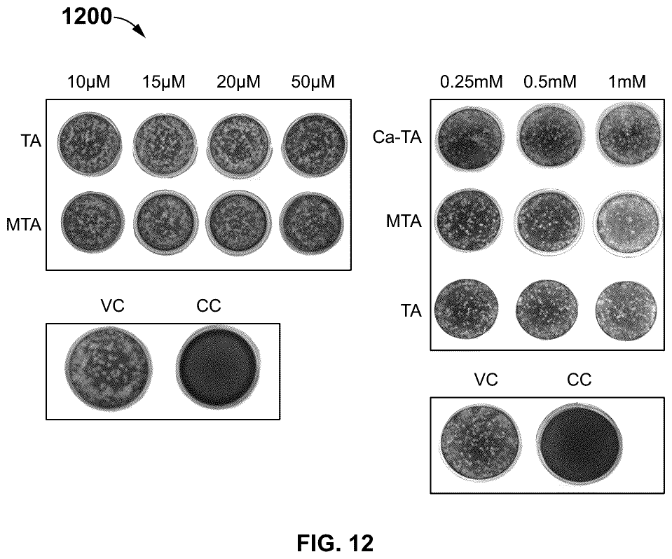

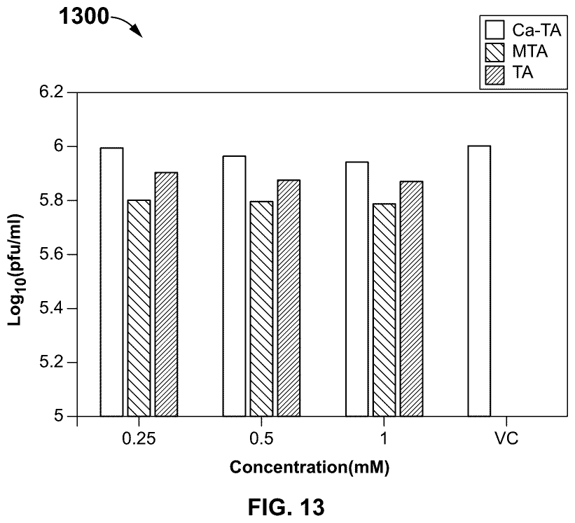

FIG. 12 exemplarily illustrates a representation 1200 of an antiviral plaque assay of Ca-TA at various concentration against the virus infected cells. FIG. 13 is a graph 1300 illustrating an activity of TA, MTA and Ca-TA at various concentration against the virus infected cells. CC refers to the cell control which consists of only cells. So, after staining with crystal violet and the imaging turns black. VC is depicted as virus control which consists of cells and virus. R2B is the type of strain of NDV (Newcastle disease virus) used for infection. After staining numerous white dots can be seen, those are called plaques. The concentration used to check the antiviral activity of TA, MTA and Ca-TA are mentioned is about 10 mM to 50 mM. At these concentrations, there is no significant difference between the virus control and treated.

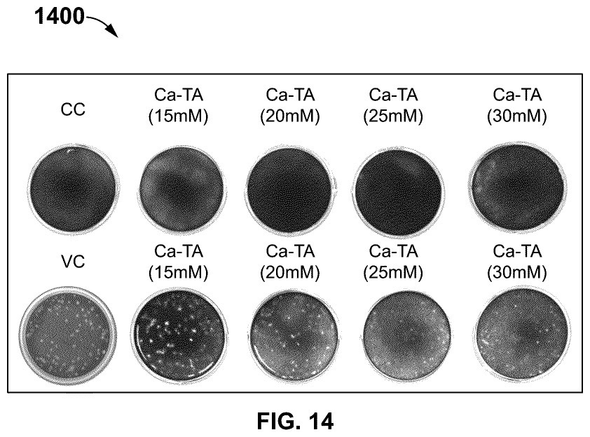

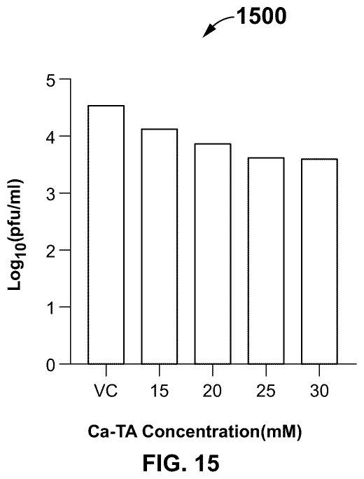

FIG. 14 exemplarily illustrates a representation 1400 of an antiviral plaque assay of Ca-TA at various concentration against the virus infected cells. FIG. 15 is a graph 1500 illustrating an activity of Ca-TA at various concentration against the virus infected cells. CC refers to the cell control which consists of only cells. So, after staining with crystal violet and the imaging turns black. VC refers to virus control which consists of cells and virus. R2B is the type of strain used for infection. After staining, numerous white dots can be seen, which represents plaques. For, cells treated with Ca-TA compound Ca-TA (15, 20, 25, 30 mM), virus is absent. Further, no toxicity was observed in these concentrations. Ca-TA is tested in the range of concentration of 15, 20, 25, 30 mM. In these wells, cells infected with virus and treated with compound at the above-mentioned concentration. Reduction in viral plaques was observed with increase in concentration of Ca-TA.

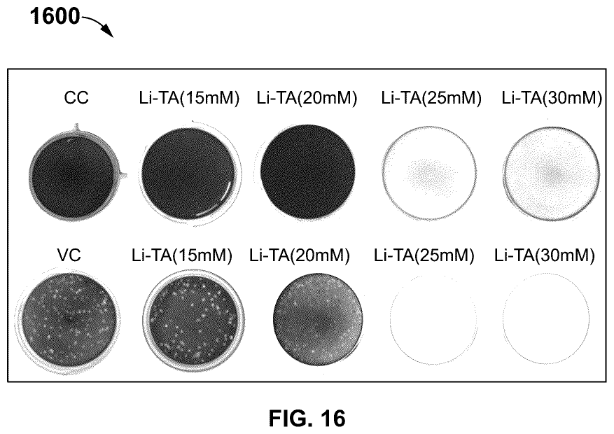

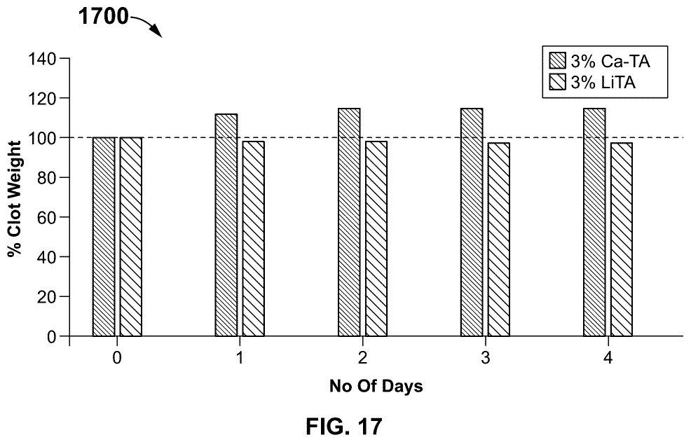

FIG. 16 exemplarily illustrates a representation 1600 of an antiviral plaque assay of Li-TA at various concentration on cells infected with virus. CC refers to the cell control which consists of only cells. So, after staining with crystal violet and the imaging turns black. VC refers to virus control which consists of cells and virus. R2B is the type of strain used for infection. After staining, numerous white dots can be seen, which represents plaques. Li-TA is tested in the range of concentration of 15, 20, 25, 30 mM. In these wells, cells infected with virus and treated with compound at the mentioned concentration. Cells treated with Li-TA compound, virus activity is reduced. FIG. 17 is a graph 1700 illustrating analysis of swelling effect for Ca-TA and Li-TA. While Ca-TA shows significant blood clot swelling, Li-TA shows essentially no swelling (any reduction is probably due to some clot drying).

### CONCLUSION

Both Ca-TA and MTA are demonstrated to exhibit protease inhibition activity. The derivatizing at NH2— eliminates or significantly reduces protease inhibition activity with modified-amido-TA (MTA). However, the derivatizing at COOH— with calcium ion retains protease inhibition activity with Ca-TA and keeps it comparable to that of TA. Ca-TA also shows comparable cytotoxicity or neurotoxicity results as that of TA. Ca-TA is predicted to indicate superior hemostasic ability as compared to TA based on the significant swelling observed with fibrin clots containing Ca-TA. MTA, shows reduced protease inhibition activity than TA, but also reduced neurotoxicity than TA based on cell-line studies. The proposed experimental cell-line work with Ca-TA demonstrates that the Ca-TA protease inhibitor has a desired antiviral efficacy against SARS-CoV-2 infections and its emerging variants.

Advantageously, the present invention uses a small molecule, preferably Ca-TA, with desired efficacy with SARS-Cov-2 system, where the inhibitor can interfere with the binding of the viral spike protein of SARS-CoV-2 to the human cell surface receptor angiotensin converting enzyme-2 (ACE-2). They demonstrate that the spike protein C-terminal domain interface contributed to a network of “hydrogen bonding and salt bridge interactions” with the ACE-2 receptor. The Ca-TA inhibitor interfere with the binding of the viral spike protein of SARS-CoV-2 to the human cell surface receptor angiotensin converting enzyme-2 (ACE-2) to avoid infections caused by SARS-CoV-2 and its emerging variants.

We claim:

1. A composition for treating viral infections, comprising:
a protease inhibitor comprising calcium salt of Tranexamic acid (Ca-TA).

2. The composition of claim 1, wherein the protease inhibitor further comprises lithium salt of Tranexamic acid (Li-TA).

3. The composition of claim 1, wherein the calcium salt of Tranexamic acid is in the range of 10 to 30 mM.

4. The composition of claim 2, wherein the lithium salt of Tranexamic acid is in the range of 10 to 30 mM.

5. The composition of claim 1, further comprises a therapeutic agent comprising antibody and peptides.

6. The composition of claim 1, further comprises a therapeutic agent comprising antibody, peptides, small drug molecules, Alzheimer's drug molecules, and combination thereof.

7. The composition of claim 1, wherein the viral infections include acute respiratory syndrome coronavirus-2 (SARS-CoV-2) and emerging variants of acute respiratory syndrome coronavirus-2 (SARS-CoV-2).

8. A method of treating viral infections, comprising the step of: administering a sustainable release composition of a protease inhibitor comprising calcium salt of Tranexamic acid (Ca-TA).

9. A composition for treating viral infections, comprising: a protease inhibitor comprising lithium salt of Tranexamic acid (Li-TA).

10. The composition of claim 9, further comprises a therapeutic agent comprising antibody, peptides and drug molecules, wherein the protease inhibitor further comprises calcium salt of Tranexamic acid (Ca-TA).

11. The composition of claim 9, used to stop bleeding in Alzheimer's disease.
